(19) United States
(12) Patent Application Publication
(10) Pub. No.: US 2008/0027550 A1
(43) Pub. Date: Jan. 31, 2008

(54) Cervical Intervertebral Disc Prosthesis Comprising An Anti-Dislocation Device And Instruments

(75) Inventors: Helmut D. Link, Hamburg (DE); Arnold Keller, Kayhude (DE)

Correspondence Address: MORRISON & FOERSTER LLP, 1650 TYSONS BOULEVARD, SUITE 400, MCLEAN, VA, 22102, US

(73) Assignee: CERVITECH, INC., Rockaway, NJ (US)

(21) Appl. No.: 10/589,899

(22) PCT Filed: Feb. 24, 2005
(86) PCT No.: PCT/EP05/01956

§ 371 Date: Aug. 18, 2006

(30) Foreign Application Priority Data
Mar. 5, 2004 (EP) 04005341.5

(51) Int. Cl. A61B 17/56; A61F 2/44

(52) U.S. Cl. 623/17.16; 606/80

(57) ABSTRACT

A cervical intervertebral prosthesis includes lower and upper anchoring plates with a prosthesis core arranged between them to create an articulated connection. The anchoring plates are designed to bear with their anchoring plate surfaces on adjacent vertebral bodies. At least one anchoring plate surface has a rib-like projection thereon which can be used to engage in the vertebral body with a form fit. In order to produce a corresponding recess in the vertebral body, an instrument having a handle, a stem, a head part and an excavating element that can be retracted into the head part may be used. This permits considerably improved securing of the cervical intervertebral prosthesis against unintended movement. The medullary canal running along the posterior margin of the vertebral column is in this way protected from damage.

### REFERENCE TO RELATED APPLICATIONS

This application is a national stage application under 35 USC 371 of International Application No. PCT/EP2005/001956, filed Feb. 24, 2005, which claims the priority of European Application No. 04 005 341.5, filed Mar. 5, 2004, the contents of both of which prior applications are incorporated herein by reference.

### FIELD OF THE INVENTION

The invention relates to a cervical intervertebral prosthesis comprising a lower anchoring plate and an upper anchoring plate, and a prosthesis core which is arranged between these and which creates an articulated connection between the anchoring plates, and also to an instrument for implanting such a prosthesis.

### BACKGROUND OF THE INVENTION

Intervertebral prostheses intended for implantation in the cervical region of the spinal column have to be positioned with the utmost precision, because of the small dimensions of the spinal column in this region. After it has been implanted and anchored in the bone, the prosthesis must not accidentally shift position. Even a very slight displacement of prosthesis parts in the dorsal direction entails a risk of affecting the spinal nerves. It is therefore of great importance to fix the intervertebral prosthesis securely in its implanted site. However, in the region of the cervical spine in particular, this is difficult because the small dimensions mean there is little space available.

It is known (WO-A-030 75 803) for the anchoring plates of the intervertebral prostheses to be provided on their ventral edge with a flange, and for this flange to be secured to the vertebral body by means of screws. To obtain a sufficiently secure connection, the screws and the flange need to have dimensions which are difficult to reconcile with the difficult implantation conditions in the region of the spinal column. This difficulty is bypassed in another construction (WO-A-030 75 804) which proposes a shortened flange without screw connection as a means of securing against slipping in the dorsal direction, and a toothed surface of the anchoring plates as a means of securing against slipping in the ventral direction. This construction is well suited for implantation in the confined conditions in the region of the cervical spine. Under certain circumstances, an increased degree of security of the connection is desirable.

In addition to the teeth, it is also known to provide self-tapping ribs that extend in the anterior-posterior direction (WO 03/075804). These ribs press automatically into the end face of the vertebral body. This does not permit any securing against undesired movement in the AP direction. Because of the self-tapping property, the rib introduces considerable forces into the anchoring plates and these forces also act partially in the horizontal direction. This increases the risk of incorrect positioning. A similar prosthesis is known from U.S. Pat. No. 6,517,580.

To improve the anchoring of the prosthesis on the vertebral bodies, it is also known for protruding pins to be provided on that surface of the anchoring plate directed toward the vertebral bodies. Difficulties arise, however, in forming suitable depressions in the vertebral body for receiving the pins. This cannot successfully be done with the required precision, so that the prosthesis often has some play. It is also known to arrange an elevation in the shape of a spherical cap on the surface of the anchoring plate (US-A-2001/0016773). Because of its rounded shape, a sufficient locking action cannot be obtained with this. An intervertebral prosthesis is also known (DE-U-203 11 400) which has anchoring projections on that surface of the anchoring plate directed toward the vertebral bodies. This prosthesis is of a different type without a sliding core, and instead of the latter it comprises convex articulation surfaces directly on the anchoring plates. The forces are thus introduced in a very concentrated way, with the result that they have to be taken up by the anchoring projections.

### SUMMARY OF THE INVENTION

The object of the invention is to improve the secure connection of a cervical intervertebral prosthesis while maintaining good implantation properties.

The solution according to the invention lies in an instrument for implanting a cervical intervertebral prosthesis including two anchoring plates and a prosthesis core arranged between them, in which the instrument includes a handle, a stem, and a head part which is arranged at an end of the instrument remote from the handle and whose dimensions are chosen such that the head part can be inserted into an implantation space that has been created between adjacent vertebral bodies for receiving the intervertebral prosthesis. The head part includes an excavating element configured for creating a recess in a cranial-caudal direction in the adjacent vertebral bodies and an actuating device provided for the excavating element which is movable between a rest position, in which the actuating device is retracted in the head part, and a working position, in which the actuating device protrudes from the head part transversely with respect to the stem.

It also lies in a cervical intervertebral prosthesis that includes a lower anchoring plate and an upper anchoring plate, each of which has an anchoring plate surface for bearing on an adjacent vertebral body, and a prosthesis core arranged between the lower and upper anchoring plates which creates an articulated connection between the anchoring plates. At least one of the two anchoring plate surfaces has a projection configured for a form-fit engagement in the vertebral body transverse to an anterior-posterior direction relative to its location of implantation.

The invention further extends to a method that includes the steps of spreading two adjacent vertebral bodies apart, working end faces of the vertebral bodies to create a seat for the cover plates, using an instrument with a head part and an excavating element which can emerge from the excavating element in a cranial-caudal direction to create a recess in the cranial-caudal direction in at least one end face of the adjacent vertebral bodies, removing the instrument and inserting the intervertebral prosthesis which, on at least one surface of the cover plates directed toward the vertebral body, has a projection engaging in the recess.

Advantageous developments are the subject matter of the detailed description below.

An instrument according to the invention for implanting a cervical intervertebral prosthesis of the type mentioned at the outset comprises a handle, a stem, and a head part which is arranged at an end remote from the handle and whose dimensions are chosen such that it can be inserted into the space that has been created for receiving the intervertebral prosthesis, the head part having an excavating element for creating a recess in the cranial-caudal direction, and an actuating device is provided for the excavating element which is movable between a rest position, in which it is retracted in the head part, and a working position, in which it protrudes from the head part transversely with respect to the stem. The instrument can be pushed with its head part toward the intended implantation site, which has been prepared in a manner known per se. When the excavating element is located in its rest position, in which it is retracted in the head part, said head part can be advanced to the intended implantation site without any difficulty. X-ray checks are expediently carried out to verify that the correct position has been reached. For this purpose, it may be expedient to provide separate X-ray markings on the head part. To create a recess on the end face of the vertebral body into which a rib-like projection arranged on the anchoring plate of the intervertebral prosthesis can engage with a form fit, the excavating element is moved into its working position. The recess can then be created by actuating the excavating element. In order to withdraw the instrument, the excavating element can then be returned to its retracted rest position. The excavating element can be driven out on one side or on both sides. The invention thus makes available an instrument, used for implanting the intervertebral prosthesis according to the invention, which can be easily advanced to the intended implantation site and which, in this position, creates recesses allowing the projections to engage with a form fit in the vertebral body.

The head part can at the same time be designed as a broach (rasp with transverse teeth), of the kind used for preparing an implant bed by removal of bone in the anterior-posterior direction in the vertebral bodies, or also as a trial prosthesis with which it is possible, by means of X-ray control, to estimate the size and position of the prosthesis that is later to be implanted.

The excavating element is expediently a cutter disk. It preferably has at least one pair of cutting fins arranged in an offset manner about the circumference. In the rest position, the cutting fins are positioned in such a way that they do not protrude from the head part. When the cutter disk is rotated via the actuating element, the cutting fins emerge from the head part perpendicular to the direction of the stem, from the cranial/caudal surfaces of the head part, and thus engage in the adjacent vertebral body. By moving the cutter disk, the recess is then created. It is expedient to arrange the cutting fins of one pair lying exactly opposite one another on the cutter disk. In this way, the recess can be produced in both adjacent vertebral bodies in the same cutting operation. This ensures that the recesses are in alignment. In addition, there are then no horizontal forces acting on the head part. Provision can also be made, however, to arrange the cutting fins so that they do not lie exactly opposite one another, but instead are offset by a certain angle which is dimensioned such that, when the cutter disk is rotated from its rest position, one cutting fin first comes into contact with one of the two adjacent vertebral bodies and cuts a recess therein, and it is only when this cutting fin has worked its way into this vertebral body that the other cutting fin emerges from the opposite cranial/caudal surface of the head part and cuts into the other of the two vertebral bodies. This has the advantage that the forces needed for the actuation are smaller, because the two vertebral bodies are not cut simultaneously, but instead after one another.

To avoid easier cutting and breaking of bone material, it is expedient to provide cutting fins of different heights. They are arranged in such a way that, during the movement from the rest position, first the cutting fin with the lower height emerges and cuts into the vertebral body, and thereafter the cutting fin with the greater height. It will be appreciated that it is also possible to provide more than two cutting fins of different height. To be able to cut simultaneously into both adjacent vertebral bodies, the cutting fins of different height are expediently arranged in pairs lying opposite one another.

However, different types of excavating elements can also be provided. In another embodiment, it is designed as a drill. A pushing/screwing drive mechanism is expediently provided for actuating it. A plurality of drills can also be provided, with at least two drills expediently being arranged transversely with respect to the stem.

The excavating element preferably has a movable axis of rotation. This permits simple actuation by means of a rotatable shaft.

The excavating element is expediently displaceable along a guide, such that a slit can be milled.

The actuating element preferably comprises a handle and a transmission shaft. Such manual actuation easily holds the instrument in place and allows the operating surgeon to move the excavating element as he sees fit. It has proven useful to provide the actuating element with an indexing means which marks the rest position. In this way, the operating surgeon can ensure that the excavating element is located in its rest position before he pushes the instrument in or withdraws it. Manual actuation is not imperative, however, and, instead, a rotary drive coupling for actuation by a motor can also be provided.

According to the invention, in a cervical intervertebral prosthesis comprising a lower anchoring plate and an upper anchoring plate which are each designed with an anchoring plate surface for bearing on an adjacent vertebral body, and comprising a prosthesis core which is arranged between these and which creates an articulated connection between the anchoring plates, provision is made that at least one of the two anchoring plate surfaces comprises a rib-like projection for form-fit engagement in the vertebral body.

The invention is based on the recognition that, by means of the rib-like projection according to the invention, it is possible to achieve a form-fit engagement of the base plate or anchoring plate on the vertebral body. The projection engaging in the vertebral body prevents undesired shifting of the anchoring plate relative to the vertebral body. It is thus possible to avoid dislocation of the cervical intervertebral prosthesis as a whole. It has been found that, with the projection engaging with a form fit in the vertebral body, it is possible to anchor the intervertebral prosthesis so securely on the vertebral body that there need be no fear of subluxation in the sense of a migration in the dorsal direction. It will be appreciated that the projection is not only able to prevent an undesired movement in the dorsal direction, but also in the opposite direction, i.e. ventrally. The operating safety of the intervertebral prosthesis provided with the projection according to the invention is thus increased considerably.

The projection can be of any desired shape. It is important, above all, that the form-fit engagement of the projection in the vertebral body is configured as an undercut in the AP direction of insertion of the intervertebral prosthesis into the space between the adjacent vertebral bodies. It has proven suitable to design the projection as a rib. It is preferably arranged in a plane parallel to the ventral and dorsal edge of the anchoring plate. In the implanted state, the rib is thus transverse to the AP direction, thereby offering the greatest possible resistance against undesired displacement. It may be expedient to make the top edge of the rib convex. It has proven useful to use a diameter of curvature of 3 to 10 mm. The insertion of the anchoring plate with its projection into its position on the vertebral body is simplified because the anchoring plate can use its convex shape to seek out its position. In addition, the convex configuration has the advantage of avoiding jagged corners protruding into the vertebral body. The danger of undesired peak loads in the corner area is thus counteracted. Finally, the convex configuration of the rib, ideally in the form of a segment of a circle, also has the advantage that the congruent recess in the vertebral body can easily be produced using a rotatable cutting tool of corresponding shape.

In another expedient embodiment, the projection is divided into two or more segments with a gap lying between them. However, when there are several segments, it is not necessary for each one to engage in its own recess. It is equally possible to use, as the recess for the form-fit engagement, one slit into which the segments arranged in one plane engage.

The projection is preferably arranged outside an edge area of the anchoring plate surface. An edge area is understood as the outer sector of the anchoring plate surfaces which takes up about 1/10 of the total surface area of the anchoring plate. In this area, the danger of the projection breaking out of its form-fit engagement in the vertebral body under high loads is reduced. At its margins, particularly in the ventral margin and dorsal margin, it is true that the vertebral body has greater strength than in the area lying in between. However, arranging the projection in the anterior or posterior area would have the disadvantage that the recess for form-fit engagement of the projection would have to be formed in a hard and brittle area of the vertebral body. There would then be a danger of bone parts splintering off. It has been found that arranging the projection so that it is slightly offset toward the dorsal direction, preferably in an area between ⅗ and ¾ of the extent in the AP direction, provides the possibility of good force transmission and also allows implantation to be carried out safely and without risk of bone splintering.

The projection can have a height of 0.3 to 0.5 mm, preferably 1.0 to 3.0 mm, above the level of the anchoring plate surface. If the latter is provided with teeth, which is of advantage for further increasing the reliability of the connection, the level of the anchoring plate surface is then defined by the top edge of the teeth. The projection is advantageously designed narrowing toward the top. This permits a self-centering effect during insertion of the anchoring plate on the vertebral body. Small inaccuracies can be compensated in this way. Implantation is made easier.

### BRIEF DESCRIPTION OF THE DRAWINGS

The invention is explained below with reference to the drawing which depicts advantageous illustrative embodiments, and in which:

FIG. 1 shows a perspective view of an illustrative embodiment of a cervical prosthesis according to the invention;

FIG. 2 shows a posterior view of the cervical prosthesis according to FIG. 1;

FIG. 3 shows a posterior-cranial view;

FIG. 4 shows a view of the posterior end of an illustrative embodiment of an instrument according to the invention;

FIG. 5 shows a cross-sectional view through a head part of the instrument according to FIG. 4;

FIG. 6 shows a cross-sectional view of an alternative illustrative embodiment of an instrument;

FIG. 7 shows the instrument according to the invention in a perspective view, obliquely from behind;

FIG. 8 shows an enlarged representation of the head part of the instrument;

FIG. 9 shows a plan view of the instrument according to the invention;

FIG. 10 shows a schematic view of two adjacent vertebral bodies between which a cervical prosthesis according to the invention is to be implanted;

FIG. 11 shows a schematic view of the two vertebral bodies according to FIG. 9 when the implantation site is being prepared;

FIG. 12 shows a further stage in the preparation according to FIG. 10; and

FIG. 13 shows the cervical prosthesis implanted between the adjacent vertebrae.

### DETAILED DESCRIPTION OF THE INVENTION

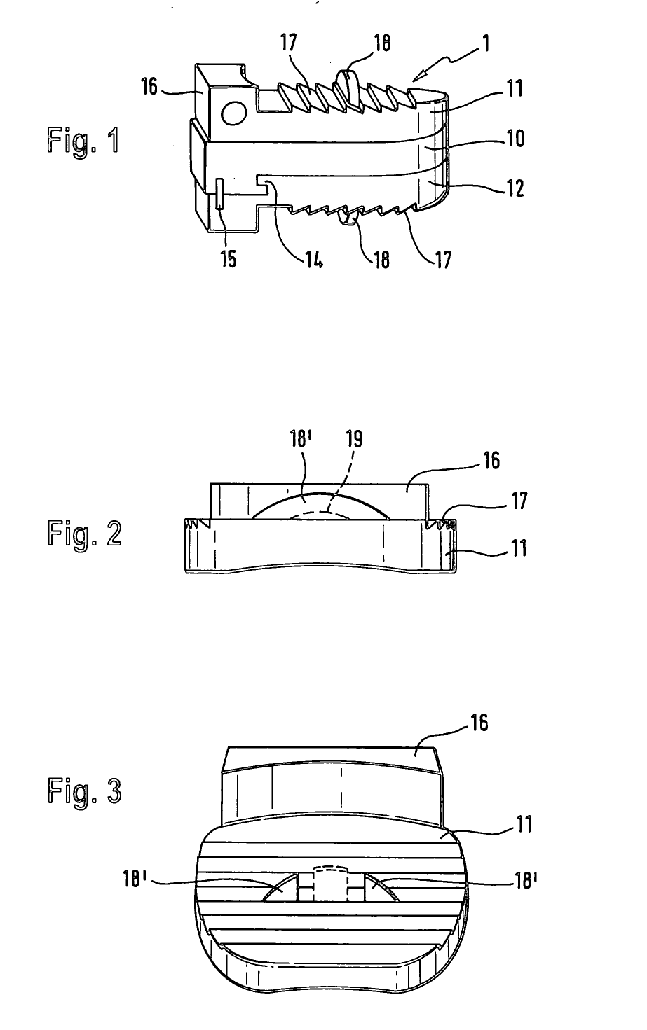

The illustrative embodiment shown in FIGS. 1 to 3 involves a cervical prosthesis according to the invention which is designated overall by reference number 1. It is provided for implantation in the space between two adjacent vertebral bodies of the cervical spine (see FIG. 13).

The cervical prosthesis 1 comprises an upper closure plate 11 and a lower closure plate 12, with a sliding core 10 arranged between them. The cervical prosthesis 1 is provided for implantation in the space between two adjacent vertebrae of the cervical spine of a human. The upper anchoring plate 11 is secured to the bottom face of the cranial vertebra, and the lower anchoring plate 12 is secured to the top face of the caudal vertebra. The anchoring plates 11, 12 are made of a hard, resistant material, in particular titanium, or another biocompatible material. At least those surfaces of the anchoring plates 11, 12 serving to bear on the adjacent vertebral bodies are preferably provided with a coating that promotes bone growth, for example hydroxyapatite. The prosthesis core 10 is made of polyethylene or of another plastic that promotes sliding and is sufficiently resistant to wear. The prosthesis core 10 is connected securely, but also releasably, to the lower anchoring plate 12. This connection is made using an undercut ledge 14 on the anterior face (on the left in FIG. 1) of the lower anchoring plate 12, into which the prosthesis core provided with a complementary groove can be pushed. When it has thus been pushed in, the prosthesis core 10 is secured by means of a small plate 15. The top of the prosthesis core 10 and the bottom of the upper anchoring plate 11 form interacting, complementary slide surfaces, which preferably have a spherical configuration.

On their anterior face, the anchoring plates 11, 12 are provided with an edge designed as a flange which protrudes in the cranial direction on the upper anchoring plate 11 and in the caudal direction on the lower anchoring plate 12. The rear face of the flange 16 pointing in the posterior direction (to the right in FIG. 1) has an abutment surface for the ventral margin of the vertebral body. To prevent the anchoring plates 11, 12 from jutting out in the ventral direction and thus possibly causing irritation of internal organs, the ventral margin of the vertebral bodies is preferably worked in such a way that a recess is formed into which the flange 16 of the anchoring plates 11, 12 is embedded. The anterior edge of the flange 16 is preferably rounded in order to ensure that the flange 16 bears securely on the vertebral body. This also provides a certain degree of protection against undesired lateral displacements. So that not too much material has to be removed from the vertebral bodies in order to provide for the recessed arrangement of the flange 16, its dimensions are expediently small. In particular, it should have only a small height above the top face of the anchoring plate 11 and below the underside of the anchoring plate 12. It should lie between 0.5 and 2 mm, preferably between 0.8 and 1.3 mm. Expressed in relation to the size of the intervertebral prosthesis, the height should measure approximately 0.5 to 2/10 of the total dimension in the anterior-posterior direction (AP direction).

The top faces of the anchoring plates 11, 12 are provided with teeth 17 across the greater part of their surface area. These have a sawtooth configuration, the steeper flank pointing anteriorly toward the flange 16 and the less steep flank pointing in the posterior direction. The steep flank of the teeth 17 preferably encloses an angle of 70 to 90 degrees with the plane of the anchoring plates 11, 12. The teeth 17 are configured such that they are oriented transverse to the AP direction. By virtue of this orientation, the teeth 17 exert a posteriorly acting force on the cervical intervertebral prosthesis 1 and thus prevent undesired displacement of the cervical prosthesis 1 in the anterior direction. The flange 16 in turn secures the cervical prosthesis 1 against movement in the posterior direction. As a result, the cervical prosthesis is therefore secured against undesired movement in both directions.

To improve the securing action and to protect against dislocation, the surface of the anchoring plates 11, 12 is provided with a cranially protruding rib or caudally protruding rib. The rib is oriented parallel to the teeth 17 and transverse to the AP direction. Its top face is configured as an arc segment of a circle. The thickness of the rib 18 is preferably constant along the entire height, although it can also narrow toward the top. A self-tapping function is not provided. The rib 18 is connected fixedly to the respective anchoring plate 11, 12 by welding or hard soldering. However, provision can also be made for the anchoring plate 11, 12 and the rib 18 to be produced in one piece.

If the ligament apparatus holding the vertebral bodies together is weakened and there is therefore a danger of the pressure applied to the cervical prosthesis 1 by the vertebral bodies being low, it may be advisable to provide the rib 18 with an aperture 19, as is indicated by the broken line in FIG. 2. The aperture 19 means that, after the anchoring plates have been implanted in the respective vertebral body, bone substance is able to grow through this aperture 19. In this way, the anchoring plate is fixed on the vertebral body in such a way that it cannot lift from the vertebral body.

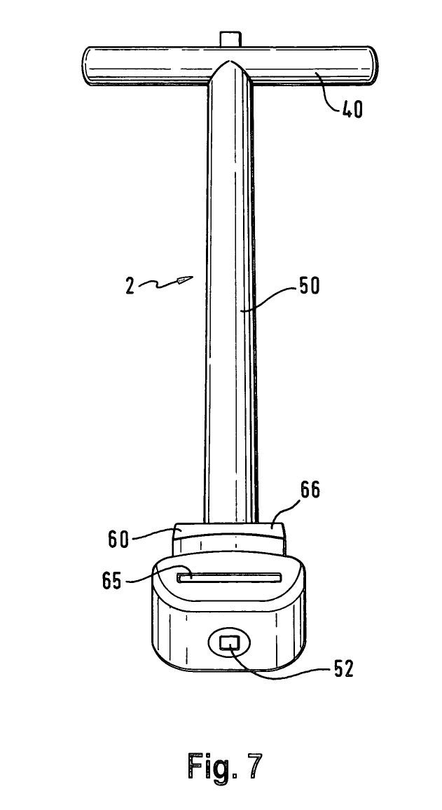

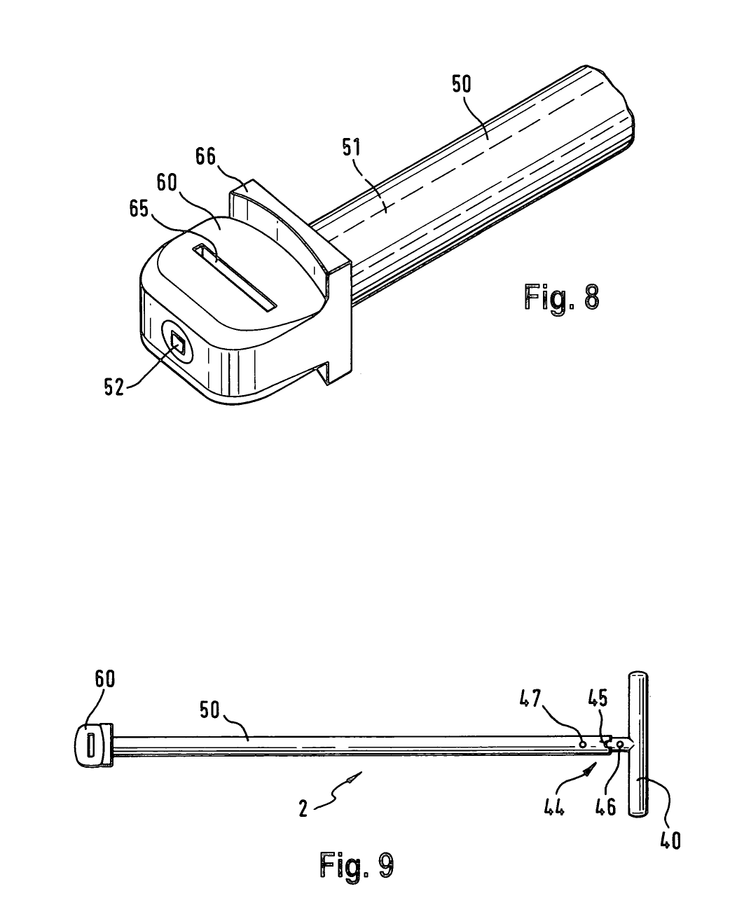

The rib 18 has a height of 1.5 mm. As has already been stated, it should not be self-tapping. It is therefore necessary, when preparing the implantation site, to work a suitable recess into the corresponding intervertebral surfaces. To do this, the instrument shown in FIGS. 3 to 9 is used. The instrument is designated in its entirety by reference number 2. It comprises a handle 40, a stem 50, and a head part 60. The head part 60 functions as an exploratory part and has the contour and dimensions of the cervical prosthesis 1 that is to be implanted. The anterior edge of the head part 60 is provided with a flange 66 corresponding to the flange 16 of the cervical prosthesis 1. The instrument can thus serve as an exploratory gauge for the cervical prosthesis I that is to be implanted.

A cutter disk 7 acting as an excavating element is arranged in the head part 60. For this purpose, the head part 60 has a slit 65 which extends along the full height of the head part from the top face 63 to the bottom face of the head part 60. The cutter disk is designed as a double-finned cutter with two pairs of fins lying opposite one another. The first pair of fins are rough-cutting fins 72 which each have a cutting edge acting in the circumferential direction. The second pair of fins are two main cutting fins 71 which lie opposite one another and have a cutting edge pointing in the same direction as in the rough-cutting fins 72, but which, when viewed in the cutting direction, are angularly set back by about 35 degrees. The cutter disk 7 has a square drive aperture 73 at its center.

Arranged on the handle 40, there is a T-shaped transmission shaft 51 which extends through the stem 50 designed as hollow cylinder and into the head part 60. At its end remote from the handle 40, the transmission shaft 51 is provided with a square entraining part 52. The transmission shaft 51 can be moved in rotation and moved longitudinally on the stem 50. An indexing arrangement 45, 46 is provided at the end of the stem 50 toward the handle. This indexing arrangement comprises a recess 45 on the edge of the stem 50 and a marking pin 46 at the end of the transmission shaft 51 toward the handle. In a rest position, the handle 40 is rotated with the transmission shaft 51 and pushed into the stem 50 such that the marking pin 46 lies in the recess 45. To move the handle 40 in its working position, the handle 40 together with the transmission shaft 51 is pulled back a distance from the edge of the stem 50 until the marking pin 46 is free of the recess 45 and the handle 40 can be rotated with the transmission shaft 51. The square entraining part 52 on the end of the transmission shaft 51 remote from the handle is designed as a square at least along the distance that the handle has to be pulled in order to free the marking pin 46 from the recess 45. This ensures that said square at all times extends across the area of the slit 65 of the head part 60, irrespective of whether the handle 40 is in its rest position or in its rotated working position.

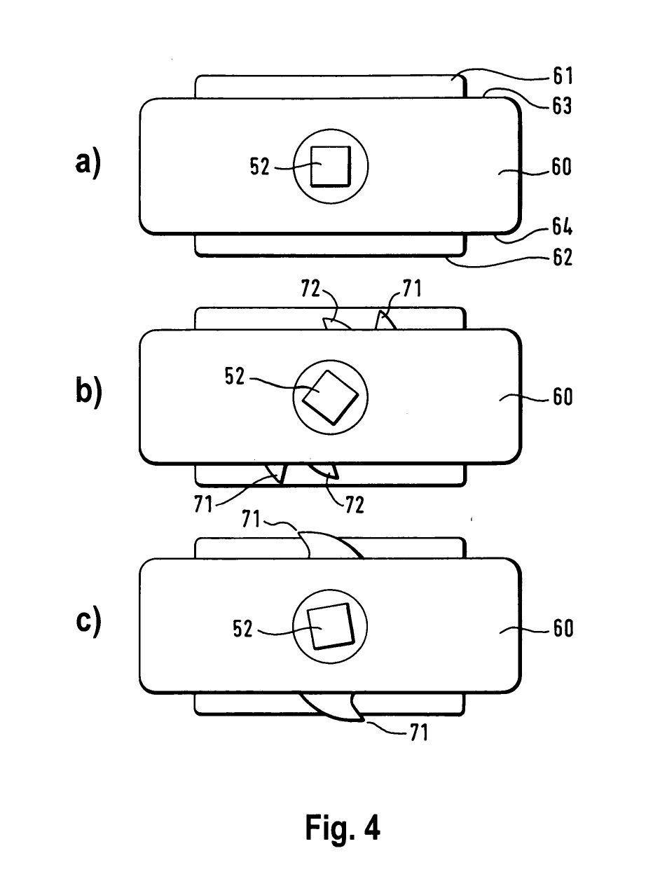

The way in which the cutter disk 7 functions is shown in detail in FIG. 4. The rest position is shown in FIG. 4a. The cutter disk 7 is located in the position in which it is also shown in FIG. 3. The cutting fins 71, 72 are retracted. In this rest position, the instrument can be pushed into the implantation space or withdrawn from it. FIGS. 4b and c show working positions. To reach this position, the handle 40 is withdrawn until the marking pin 46 is free of the recess 45 on the stem 50. The handle 40 can then be moved in the direction in which the cutting edges of the cutting fins 71, 72 point. By rotation of the cutter disk 7, the cutting fins 71, 72 move on a circular path.

First, the rough-cutting fins 72 leave the slit 65 of the head part 60 and cut a first, low slit into the adjacent face of the vertebral body. The rough-cutting fins are configured such that they break through the relatively hard margin of the vertebral body. Thereafter, the main cutting fins 71 emerge from the slit 65 and cut a larger slit in what is by comparison the softer bone substance of the vertebral body. The rotation takes place until the main cutting fins 71 on the opposite side start again to travel into the slit 65 of the head part 60. If so desired, the procedure can be repeated. By virtue of the symmetrical configuration of the cutter disk 7, the slits are cut simultaneously in the upper and lower vertebral bodies. If, in order to reduce the acting forces, this is not desired, the fins can either be provided on just one side or they are offset from one another by an angle different than 180 degrees, so that initially one set of rough-cutting fins and main cutting fins 71, 72 emerges from the slit 65, while the other set only follows later.

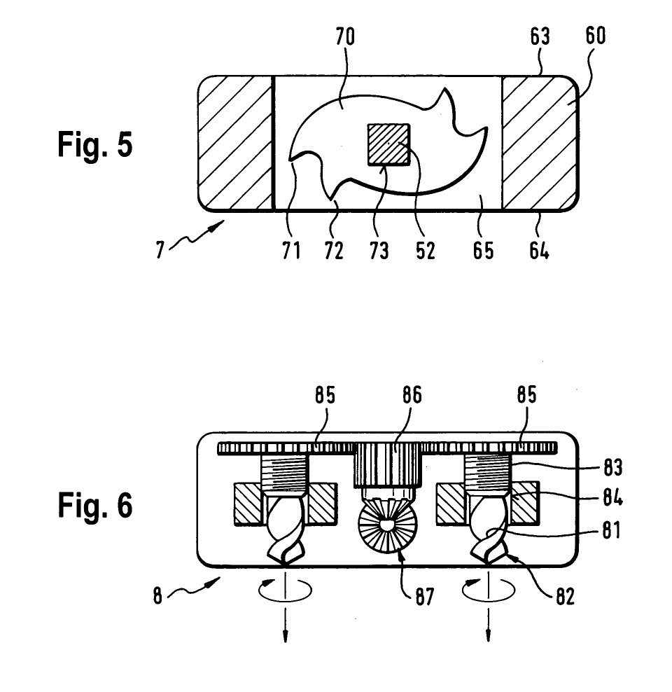

FIG. 5 shows another illustrative embodiment of an excavating element. This is a combined drilling/milling device 8. It comprises two drills 82 which are arranged in the head part 60 and transverse to the direction of the stem 50. The drills 82 have cutting rifles 81 in their lower portion. In their upper portion, they are provided with an external thread 83. The latter is guided in a matching thread 84 arranged fixedly in the head part 60. A driven toothed wheel 85 is arranged at the top end of the drill 82. It meshes with a drive wheel 86, which in turn is driven by the transmission shaft 51 via a right-angle gear drive 87. The drive wheel 86 has a greater thickness than the driven wheel 85. Its thickness preferably corresponds to the intended drill travel, i.e. to the depth of the recesses to be produced using the drills 82. A drill arrangement is provided in mirror symmetry on the opposite side. The drilling/milling device 8 functions as follows. By means of a preferably machine drive, the transmission shaft 51 is set in rotation, as a result of which the drive wheel 86 likewise turns via the right-angle gear drive 87. In their rest position, the drills are located in the position shown in FIG. 5, where the driven wheel 85 engages in the drive wheel 86 at the upper edge of the latter. By means of the rotation of the drive wheel 86, the driven wheel 85 is also turned, by which means the drill 82 is set in rotation. By means of the rotation movement, the drill 82 turns with its thread 83 into the external thread 84, as a result of which the drill 82 is moved downward. The drill 82 thus works its way with its cutting rifles 81 into the bone substance of the vertebral body. By virtue of the downwardly directed pushing movement of the drill 82, the driven wheel 85 also moves downward, always remaining in engagement with the drive wheel 86. For this purpose, the latter has a thickness which is at least as great as the travel of the drill 82. When the desired depth of the recesses is reached, reversing the direction of rotation has the effect that the drills 82 travel back to their rest position.

In an alternative embodiment, the rib 18 can also be divided into segments 18′ (see FIG. 3). The segmentation has the advantage that the resulting side surfaces avoid additional securing of the prosthesis against undesired displacements in the transverse axis. The rib 18 can be divided into two or three segments 18′, as is indicated by the broken line in FIG. 3. To improve the hold on the vertebral body, it is possible that the recess provided in the vertebral body for receiving the rib segments 18′ is also segmented instead of being continuous. More details on this are given in connection with the drilling/milling device 8.

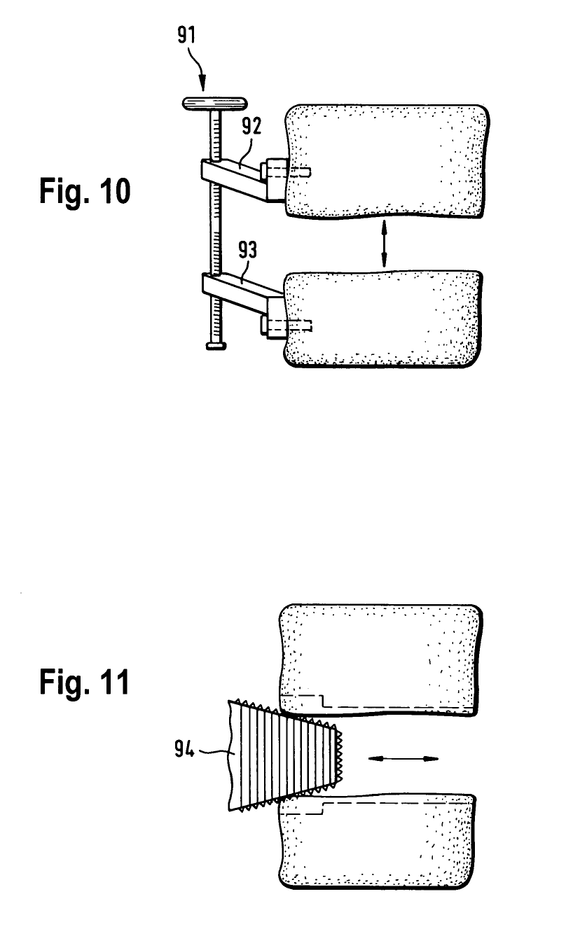

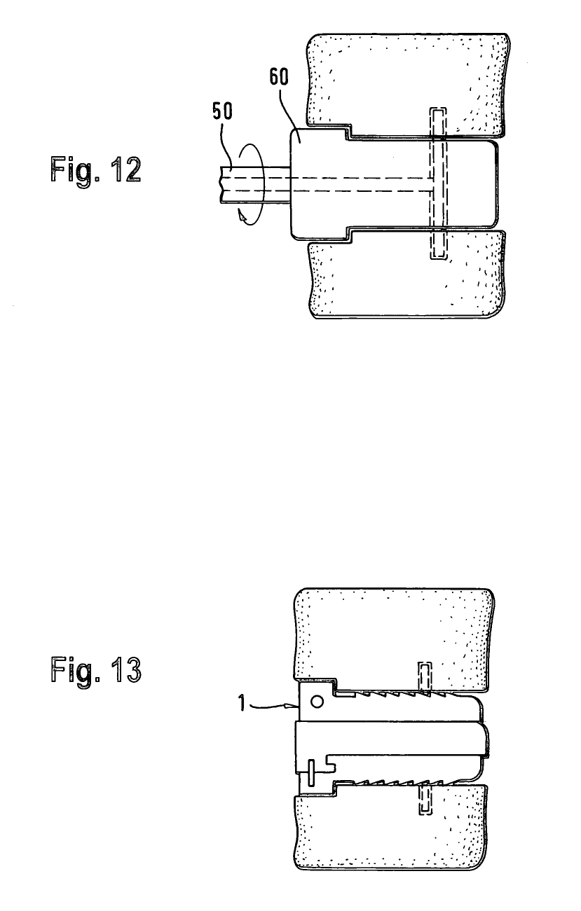

The method for implanting the prostheses according to the invention using the tool according to the invention can be explained as follows. In a first step, the adjacent vertebral bodies between which the cervical prosthesis 1 is to be implanted are prepared for receiving a retractor 91. This is done by means of the legs 92, 93 of the retractor 91 being secured on the anterior face of the two vertebral bodies by screw connections. The retractor 91 has an angled design to ensure that the immediate access area from the anterior direction into the space remains free. After the vertebrae have been spread to the desired distance apart, the space between them is prepared for receiving the cervical prosthesis 1. This is done by excavating excess bone substance in order to create a suitable bearing surface for the anchoring plates 11, 12 and for the flange 16 (see FIG. 10). After the implantation site has been prepared thus far, the instrument 2 according to the invention is applied. The head part 60 is pushed into the prepared intervertebral space. By actuating the handle 40, the cutter disk 7 is activated, so that the cutting fins 71, 72 cut a recess for the rib 18 into the cranially adjacent and caudally adjacent vertebral bodies. Thereafter, the cutter disk 7 is guided back to its rest position, and the instrument 2 can be withdrawn. The preparatory work is thus completed. The cervical prosthesis 1 can now be fitted, the vertebral bodies possibly being spread slightly farther apart by means of the retractor 91 in order to provide sufficient space for insertion of the ribs 18 into the recesses. After removal of the retractor 91, the implantation is complete.

1-22. (canceled)

23. An instrument for implanting a cervical intervertebral prosthesis including two anchoring plates and a prosthesis core arranged between them, the instrument comprising a handle, a stem, and a head part which is arranged at an end of the instrument remote from the handle and whose dimensions are chosen such that the head part can be inserted into an implantation space that has been created between adjacent vertebral bodies for receiving the intervertebral prosthesis, the head part comprising an excavating element configured for creating a recess in a cranial-caudal direction in the adjacent vertebral bodies and an actuating device provided for the excavating element which is movable between a rest position, in which the actuating device is retracted in the head part, and a working position, in which the actuating device protrudes from the head part transversely with respect to the stem.

24. The instrument as claimed in claim 23, wherein the excavating element is a cutter disk.

25. The instrument as claimed in claim 24, wherein the cutter disk has at least one pair of cutting fins arranged in an offset manner about its circumference.

26. The instrument as claimed in claim 25, wherein the cutting fins have different heights.

27. The instrument as claimed in claim 25 or 26, wherein the cutting fins are arranged in pairs lying opposite one another.

28. The instrument as claimed in claim 23, wherein the excavating element is a drill.

29. The instrument as claimed in claim 28, further comprising a pushing/screwing drive mechanism configured for actuating the drill.

30. The instrument as claimed in claim 28 or 29, wherein at least two drills are arranged transversely with respect to the stem.

31. The instrument as claimed in claim 28 or 29, wherein the excavating element has a spherical cutter section.

32. The instrument as claimed in claim 23, 24, 25, 26, 28 or 29, wherein the excavating element is longitudinally movable along a guide.

33. The instrument as claimed in claim 23, 24, 25, 26, 28 or 29, wherein the actuating element comprises a handle and a transmission shaft.

34. The instrument as claimed in claim 23, 24, 25, 26, 28 or 29, wherein the actuating element has a rotary drive coupling.

35. A cervical intervertebral prosthesis, comprising a lower anchoring plate and an upper anchoring plate, each of which has an anchoring plate surface for bearing on an adjacent vertebral body, and a prosthesis core arranged between the lower and upper anchoring plates which creates an articulated connection between the anchoring plates,
wherein at least one of the two anchoring plate surfaces comprises a projection configured for a form-fit engagement in the vertebral body transverse to an anterior-posterior direction relative to its location of implantation.

36. The cervical intervertebral prosthesis as claimed in claim 35, wherein the projection is arranged outside an edge area of at east one of the anchoring plate surfaces.

37. The cervical intervertebral prosthesis as claimed in claim 36, wherein the projection is offset from the center of its anchoring plate surface in posterior direction relative to the location of implantation in an area between ⅗ and ¾ of the extent of the anchoring plate surface in the anterior-posterior direction.

38. The cervical intervertebral prosthesis as claimed in claim 35, 36 or 37, wherein the projection has a height of 0.3 to 5.0 mm above the level of the anchoring plate surface.

39. The cervical intervertebral prosthesis as claimed in claim 38, wherein the projection has a height of 1.0 to 3.0 mm above the level of the anchoring plate surface.

40. The cervical intervertebral prosthesis as claimed in claim 36, 37 or 38, wherein the projection has a spherical section.

41. The cervical intervertebral prosthesis as claimed in claim 36, 37 or 38, wherein the projection is divided into two or more segments with a gap lying between the segments.

42. The cervical intervertebral prosthesis as claimed in claim 41, wherein the segments are configured in the shape of a bolt.

43. A method for implanting a cervical intervertebral prosthesis comprising two cover plates with a prosthesis core arranged therebetween, the method comprising:
a) spreading two adjacent vertebral bodies apart,
b) working end faces of the vertebral bodies to create a seat for the cover plates,
c) using an instrument with a head part and an excavating element which can emerge from the excavating element in a cranial-caudal direction to create a recess in the cranial-caudal direction in at least one end face of the adjacent vertebral bodies,
d) removing the instrument and inserting the intervertebral prosthesis which, on at least one surface of the cover plates directed toward the vertebral body, has a projection engaging in the recess.

44. The method as claimed in claim 43, wherein the instrument further comprises a handle and a stem, and the head part is arranged at an end of the instrument remote from the handle and has dimensions are chosen such that the head part can be inserted into the recess, the head part further comprising an actuating device provided for the excavating element which is movable between a rest position, in which the actuating device is retracted in the head part, and a working position, in which the actuating device protrudes from the head part transversely with respect to the stem.

45. The method as claimed in claim 43 or 44, wherein the intervertebral prosthesis comprises a lower anchoring plate and an upper anchoring plate, each of which has an anchoring plate surface for bearing on an adjacent vertebral body, and a prosthesis core arranged between the lower and upper anchoring plates which creates an articulated connection between the anchoring plates,
wherein at least one of the two anchoring plate surfaces comprises a projection configured for a form-fit engagement in the vertebral body transverse to an anterior-posterior direction relative to its location of implantation.
